(19) United States
(12) Patent Application Publication
(10) Pub. No.: US 2025/0387527 A1
(43) Pub. Date: Dec. 25, 2025

(54) SANITIZING AND DISINFECTING SUBSTRATES LOADED WITH HYPOCHLOROUS ACID SOLUTION

(71) Applicants: Marion E. Paris, Jr., Lincolnton, NC (US); Dianne B. Ellis, Cary, NC (US)

(72) Inventors: Marion E. Paris, Jr., Lincolnton, NC (US); Dianne B. Ellis, Cary, NC (US)

(21) Appl. No.: 19/327,289

(22) Filed: Sep. 12, 2025

Related U.S. Application Data
(63) Continuation-in-part of application No. 17/721,867, filed on Apr. 15, 2022.
(60) Provisional application No. 63/217,738, filed on Jul. 1, 2021.

(51) Int. Cl. A61L 2/18; A01N 25/10; A01N 59/00; A01P 1/00; A61L 101/06; B08B 1/14; B32B 5/02; B32B 5/26; D04H 1/4291; D04H 1/435; D04H 1/4374; D04H 1/49; D04H 3/16

(52) U.S. Cl. CPC A61L 2/18; A01N 25/10; A01N 59/00; A01P 1/00; B08B 1/143; B32B 5/022; B32B 5/267; D04H 1/4291; D04H 1/435; D04H 1/4374; D04H 1/49; D04H 3/16; A61L 2101/06; A61L 2202/17; B32B 2250/03; B32B 2250/20; B32B 2262/0253; B32B 2262/0284; B32B 2307/73; D10B 2321/022; D10B 2331/04; D10B 2509/026

(57) ABSTRACT

Sanitizing and disinfecting substrates loaded with, or to be loaded with a stable liquid HOCl solution are disclosed. The substrates may be a multi-ply laminate of a nonwoven fabric material having first and second outer layers of a normally hydrophobic synthetic polymer polypropylene (PP), polyethylene (PE) and/or polyester (PET) fibers, and an inner layer of synthetic polymer PET fibers and/or organic polymer fluff pulp. The layers may be bonded together by adhesive bonding, chemical bonding or thermal bonding. A container is provided for receiving and storing the substrates and for subsequently dispensing the substrates. A combination container and substrates is provided for receiving and storing the substrates and subsequently dispensing substrates loaded with the HOCl solution or to be loaded with the HOCl solution. Substrates may be pre-moistened with the HOCl solution and disposed within the container, or alternatively, disposed within the container and subsequently moistened with the HOCl solution.

### CROSS REFERENCE TO RELATED APPLICATION

This United States utility patent application is a continuation-in-part application of U.S. application Ser. No. 17/721,867, filed on Apr. 15, 2022, the entire content of which is hereby incorporated herein to the fullest extent of the law.

### FIELD OF THE INVENTION

The present invention relates generally to sanitizing and disinfecting substrates loaded with, or to be loaded with, a sanitizing and disinfecting solution. In advantageous embodiments, the invention is a substrate in the form of a nonwoven fabric material loaded with a hypochlorous acid (HOCl) solution for sanitizing and disinfecting a hard surface. In one advantageous embodiment, the substrate is a HOCl applicator in the form of a wipe. In another advantageous embodiment, the substrate is a HOCl applicator in the form of a continuous roll disposed within a canister that is subsequently filled with a HOCl solution.

### BACKGROUND OF THE INVENTION

Many facilities, including hospitals, nursing homes, prisons and schools, are highly susceptible to multi-drug resistant organisms (MDROs), commonly referred to as infectious bacteria and viruses. For example, the Centers for Disease Control and Prevention (CDCP) estimates that infections acquired from healthcare and food service facilities kill more individuals each year than vehicle accidents, breast cancer or AIDS. Consequently, the Environmental Protection Agency (EPA) and the Food and Drug Administration (FDA) mandate effective cleaning, sanitizing and disinfecting products and procedures to be used in facilities that provide healthcare services and/or food services. In response, hospitals, nursing homes, prisons and schools have instituted detailed cleaning, sanitizing and disinfecting protocols along with intensive training programs for environmental services personnel to ensure that areas accessed by patients, staff and the public are clean, hygienic and free of infectious bacteria and viruses.

Such facilities, especially healthcare facilities, have historically utilized a variety of high, medium and low level cleaning, sanitizing and disinfecting solutions, including formaldehyde, hydrogen peroxide, peracetic acid, chlorine-releasing agents (CRAs), such as sodium hypochlorite, chlorine dioxide, etc., iodophores, phenols and bis-phelols solutions. More recently, solutions of hypochlorous acid (HOCl) have been introduced as an effective and environmentally friendly alternative to traditional sanitizers and disinfectants. HOCl is a weak acid formed when chlorine dissolves in water and partially dissociates. As a result, HOCl acts as an oxidizer and a primary sanitizing and disinfecting agent in a chlorine solution. The beneficial characteristics attributed to HOCl include that it is a highly effective mechanism for destroying infectious bacteria and viruses, most notably C. diff, E-Coli, MRSA (Staph), Salmonella, Tuberculosis, Human Immunodeficiency Virus (HIV), and Severe Acute Respiratory Syndrome (SARS).

Despite being highly effective, HOCl is relatively harmless to humans at concentrations suitable for sanitizing and disinfecting. Thus, HOCl solutions are approved for use as sanitizing and disinfecting agents in hospitals, nursing homes, prisons and schools (collectively referred to herein as “institutional facilities”).

Other sanitizing and disinfecting agents commonly used in institutional facilities are not as environmentally friendly and effective as HOCl in destroying harmful bacteria and viruses. As a result, it is not uncommon for individuals to contract serious illnesses from the bacteria and viruses at institutional facilities that are treated with other sanitizers and disinfectants. The inability of other sanitizers and disinfectants to destroy infectious organisms increases healthcare costs and causes physical harm to individuals that would have been prevented with the use of a more effective HOCl sanitizing and disinfecting solution. The COVID 19 pandemic increased awareness for cleaning, sanitizing and disinfecting hard surfaces in commercial businesses, public areas and private spaces in addition to the institutional facilities that are already utilizing highly effective HOCl solutions to combat harmful bacteria and viruses.

Despite being highly effective, HOCl has a limited lifespan of effectiveness, commercially referred to as “shelf life.” Over time HOCl decomposes to chloric acid, hydrochloric acid, and oxygen; none of which separately exhibits the same desirable sanitizing and disinfecting properties as a full strength HOCl solution. The shelf life for a HOCl solution as a sanitizing and disinfecting agent is limited from the time it is produced based on its free available chlorine (FAC) concentration measured in parts per million (ppm). As used herein, the term “free available chlorine” or “FAC” is intended to mean the portion of total chlorine in the solution that is present as HOCl or hypochlorite ion (OCI—). Consequently, it is imperative to take steps to ensure that an effective HOCl solution is being used in a recommended cleaning, sanitizing and disinfecting protocol at institutional facilities, commercial businesses, public areas and private spaces. Specifically, it is essential that the HOCl solution is used within its shelf life acceptable for effective cleaning, sanitizing or disinfecting.

Unfortunately, due to its limited shelf-life, HOCl is not presently commercially available in a convenient and stable form for effective sanitizing and disinfecting. HOCl solution is typically generated in bulk by electrolysis using a relatively expensive and time consuming Electrochemically Activated Water (EAW) process. A portion of the HOCl solution is then transferred into a smaller container for sanitizing and disinfecting, such as a spray cart, spray bottle or liquid dispenser. The production date of the HOCl solution is noted on the smaller container to confirm that the HOCl solution is within its effective shelf life. The HOCl solution should be applied directly onto a hard surface and allowed to remain a predetermined dwell time for effective sanitizing and disinfecting. Oftentimes, the HOCl solution is sprayed or poured onto the hard surface and then wiped with a cloth or towel to apply the solution evenly over the hard surface. It would be more convenient and less labor intensive if the cloth or towel were pre-moistened with the HOCl solution-similar to a conventional alcohol wipe. However, until now there has been no substrate material known to be suitable for providing a HOCl solution in a more convenient form, and particularly, in the form of a pre-moistened cloth, towel or wipe (collectively referred to herein as an “applicator” or simply “applicator”) loaded with a stable HOCl solution.

It is likely that no suitable substrate material for use as an HOCl applicator has been found to date because a liquid HOCl solution does not adequately wet commonly used synthetic polymer substrate materials, such as polyester (PET), polyethylene (PE), polypropylene (PP), polyvinylchloride (PVC), nylon, etc., which are normally hydrophobic materials. As used herein, the term “hydrophobic” is intended to mean that the substrate material repels a liquid HOCl solution, and thus, does not absorb the HOCl solution. In contrast, a hydrophilic substrate material, as the term is used herein, absorbs a liquid HOCl solution. As taught by United States Patent Application Publication No. 2016/0330969 to O'Connell, a normally hydrophobic substrate material (e.g., PET, PP, PE, PVC, nylon, etc.) can only be made to be hydrophilic with the addition of a surfactant additive or by a matrix of hydrophobic and hydrophilic polymer fibers.

Conversely, a hydrophilic material, such as an organic polymer substrate material (e.g., cellulose) or a synthetic polymer substrate material (e.g., polyester) made hydrophilic, absorbs a liquid HOCl solution. However, the HOCl solution attacks and denigrates most organic substrate materials, and/or any pulp, filler, binder, adhesive or similar agent applied to a synthetic substrate material. Regardless, a substrate, and particularly at least the outer surface of the substrate, for a liquid HOCl solution needs to be hydrophobic since a hydrophilic substrate material necessarily destroys the hydrogen bonding of a liquid HOCl solution. A hydrophilic substrate material competes for and replaces the hydrogen-bonding network of HOCL because the hydrophilic surface has a stronger affinity for the hydrogen molecules than the hydrogen bonds that HOCl forms with itself.

In addition, a suitable applicator loaded with an effective HOCl solution needs to be stored within and subsequently dispensed from a container that retains the shelf life of the HOCl solution until the applicator is removed from the container and used for sanitizing and/or disinfecting a hard surface. Previous attempts to provide a suitable substrate material loaded with a shelf-stable HOCl solution for sanitizing and disinfecting a hard surface in the form of an applicator have proven unsuccessful, primarily due to the inherent chemistry of HOCl solutions that are not stable over an acceptable shelf-life. A system and method for producing a sufficiently stable HOCl solution having an acceptable shelf-life for effective sanitizing and disinfecting using a conical cell technology is disclosed in U.S. patent application Nos. 17/344,951 and 18/975,428, the entire disclosures of which are incorporated herein by reference.

In view of the foregoing, it is apparent a need exists for improved sanitizing and disinfecting substrates loaded with, or to be loaded with, an effective sanitizing and disinfecting solution. A specific need exists for sanitizing and disinfecting substrates loaded with, or to be loaded with, a stable HOCl solution for sanitizing and/or disinfecting a hard surface. Such a substrate would provide a more convenient form of an applicator, or carrier, for effectively sanitizing and disinfecting hard surfaces in institutional facilities, commercial businesses, public areas and private spaces with a stable HOCl solution having an acceptable shelf-life.

Certain aspects, objects, features and advantages of the invention will be made apparent or will be readily understood and appreciated by those skilled in the relevant art, with reference to the exemplary embodiments of the invention described herein and shown in the accompanying drawing figures. It is intended that the certain aspects, objects, features and advantages of the invention set forth herein be construed in accordance with the ordinary and customary meaning of the elements, terms and limitations of the appended claims given their broadest reasonable interpretation consistent with this written disclosure and the accompanying drawing figures, unless a further, different or contrary meaning is provided by this disclosure.

Some or all aspects, objects, features and advantages of the invention, as well as others not expressly or inherently disclosed herein, may be accomplished by one or more of the exemplary embodiments described herein and shown in the accompanying drawing figures. However, it should be appreciated that the written description and drawing figures are for illustrative purposes only, and that many modifications, substitutions or revisions may be made to the exemplary embodiments without departing from the general concepts of the invention and the intended broad scope and proper construction of the appended claims.

### BRIEF DESCRIPTION OF THE DRAWING FIGURES

The aforementioned aspects, objects, features and advantages of the invention will be more fully understood and appreciated when considered with reference to the accompanying drawing figures, wherein like reference characters designate the same or similar parts throughout the several views.

FIG. 1 is a schematic cross-section view of an exemplary embodiment of a sanitizing and disinfecting substrate according to the invention.

FIG. 2 is a perspective view of an exemplary embodiment of a first container for storing and subsequently dispensing sanitizing and disinfecting substrates according to the invention.

FIG. 3 is a perspective view of an exemplary embodiment of a second container for storing and subsequently dispensing sanitizing and disinfecting substrates according to the invention.

FIG. 4 is an exploded perspective view of an exemplary embodiment of a third container for storing and subsequently dispensing sanitizing and disinfecting substrates according to the invention.

### DETAILED DESCRIPTION OF EXEMPLARY EMBODIMENTS

Exemplary embodiments of the present invention are described in greater detail herein and shown in the accompanying drawing figures. The exemplary embodiments described and shown herein are directed to improved sanitizing and disinfecting substrates loaded with, or to be loaded with, an effective sanitizing and disinfecting solution. Advantageous exemplary embodiments described and shown herein are sanitizing and disinfecting substrates loaded with, or to be loaded with, a stable HOCl solution that are stored within and dispensed from a container that maintains an acceptable shelf-life of the HOCl solution. Various aspects, objects, features and advantages of the invention are illustrated by exemplary embodiments of suitable substrates in the form of a carrier, also referred to herein as an applicator, loaded with, or configured to be loaded with, a liquid HOCl solution, and disposed within a suitable container for storing and dispensing the substrate. In an advantageous embodiment, a substrate according to the invention comprises a multi-layer, nonwoven fabric material loaded with a stable HOCl solution in the form of a “wet wipe” for sanitizing and/or disinfecting a hard surface, as will be described in greater detail hereafter. In another advantageous embodiment, the substrate is in the form of a continuous roll, commonly referred to as a “log,” configured to be stored in a container and subsequently loaded with the HOCl solution for sanitizing and/or disinfecting a hard surface.

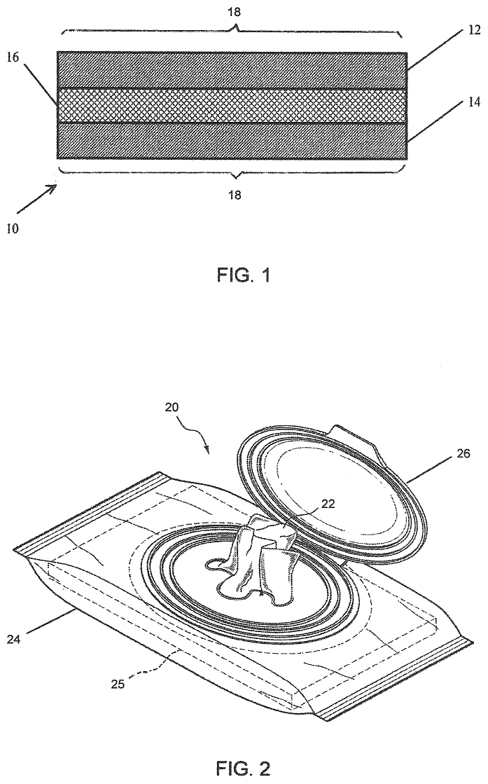

FIG. 1 is a schematic cross-section of an exemplary embodiment of a substrate 10 according to the invention. The substrate 10 is useful, by way of example and not limitation, for effectively sanitizing and/or disinfecting hard surfaces in institutional facilities, commercial businesses, public areas and private spaces with a stable HOCl solution having an acceptable shelf-life. In one aspect, a suitable substrate 10 comprises a nonwoven fabric material. Substrate 10 may comprise a synthetic polymer nonwoven fabric material having one or more layers of plastic fibers, such as high molecular weight polyolefin fibers like polypropylene (PP) or polyethylene (PE), polyester fibers like (PET) or polyethylene terephthalate (PETE), polyurethane (PUR) fibers and/or polyamide fibers. Conversely, in another embodiment the substrate 10 may be an organic polymer nonwoven fabric material, for example made from cellulosic fibers, such as wood pulp, cotton and linen. As will be readily appreciated by those of ordinary skill in the art, synthetic fabrics are normally hydrophobic rather than hydrophilic, as compared with organic fabrics. In addition to being normally hydrophobic, synthetic polymer fabrics made of plastic fibers are more resistant to degradation from caustic chemicals, such as chlorine, and in the present case, from the FAC in a sanitizing and disinfecting liquid HOCl solution.

Consequently, it is desirable that at least an outer surface of the substrate for applying the HOCl solution to a hard surface comprises a normally hydrophobic material that allows the liquid HOCl solution to adhere to the outer surface without the outer surface absorbing the HOCl solution. In other words, the outer surface adsorbs the HOCl solution but does not absorb the HOCl solution or wick the HOCl solution to an inner layer of the substrate comprising a naturally hydrophilic organic polymer material or a naturally hydrophilic filler, binder, adhesive or similar agent applied to a synthetic polymer or organic polymer material. A substrate, and particularly at least the outer surface of the substrate, for a liquid HOCl solution needs to be hydrophobic since a naturally hydrophilic substrate material necessarily destroys the hydrogen bonding of the liquid HOCl solution. A naturally hydrophilic substrate material competes for and replaces the hydrogen-bonding network of the HOCL solution because the hydrophilic surface has a stronger affinity for the hydrogen molecules than the hydrogen bonds that HOCl forms with itself.

As illustrated in FIG. 1 by way of example and not limitation, the substrate 10 may be a multi-ply laminate of a nonwoven fabric material comprising a first outer layer 12 of a normally hydrophobic Spunbond synthetic polymer and a second outer layer 14 of a normally hydrophobic Spunbond synthetic polymer, with an inner layer 16 of a Meltblown synthetic polymer or organic polymer disposed between the first and second outer layers 12, 14. As such, the tri-ply laminate nonwoven fabric substrate 10 depicted in FIG. 1 is typically referred to as Spunbond Meltblown Spunbond (SMS) fabric. In an embodiment, the substrate 10 may comprise a first outer layer 12 of normally hydrophobic Spunbond polypropylene, polyethylene and/or polyester, a second outer layer 14 of normally hydrophobic Spunbond polypropylene, polyethylene and/or polyester, and an inner layer 16 of Meltblown polypropylene, polyethylene and/or polyester disposed between the first and second outer layers 12, 14. The outer surfaces of the first and second outer layers 12, 14 may be provided with a soft calendar pattern for a purpose to be described hereafter.

In another embodiment, the substrate 10 may be a nonwoven fabric formed from a hydroentanglement bonding process by carding, air-laying or wet-laying synthetic polymer and/or organic polymer fibers. A suitable substrate 10 is a nonwoven fabric commercially available from Berry Global, Inc. of Evansville, Indiana, USA, under the tradename Spinlace®. The Spinlace® nonwoven fabric is formed from Spunbond thermoplastic fibers, such as polypropylene (PP), imaged via a proprietary Apex® technology that is specially designed and formulated for premium applications in the healthcare, hygiene, household cleaning, food service, and industrial wipes markets. By way of example and not limitation, the nonwoven fabric may be a tri-ply laminate comprising first and second outer layers 12, 14 of hydroentangled synthetic polymer Spunbond polypropylene (PP) fibers, and an inner layer 16 of organic polymer pulp, such as RISI fluff pulp. In another example, the first and second outer layers 12, 14 may be hydroentangled synthetic polymer Spunbond polypropylene (PP) fibers and the inner layer 16 may be synthetic polymer polyester (PET) fibers. In yet another example, the first and second outer layers 12, 14 may be synthetic polymer Spunbond polyester (PET) fibers and the inner layer 16 may be organic polymer pulp, such as RISI fluff pulp.

In another embodiment, the substrate 10 may be a nonwoven fabric material formed from one or more layers of synthetic polymer polyester (PET) fibers. In general, it is believed that a substrate 10 comprising a synthetic polymer PET nonwoven fabric is particularly well suited for use with HOCl solutions due to the relatively inert nature of the polyester (PET) fibers. In further embodiments, the nonwoven fabric material may comprise synthetic polymer polyester (PET) fibers, wettable PET fibers, or both, with or without a binder system. “Wettable” PET is a modified form of the synthetic polymer PET material that has been treated (e.g., with a surfactant or the like) to become hydrophilic as opposed to the normally hydrophobic standard PET. In an embodiment, the synthetic nonwoven fabric material comprises 50% PET and 50% wettable PET and is commercially known as Spunlace available from Berry Global, Inc. The basis weight may range from about 25 grams per square meter (gsm) to about 80 grams per square meter (g/sm) with an absorbency capacity of at least 500% and with a hand feel more comparable to a viscose semi-synthetic material than a PET synthetic material when wet or otherwise activated. In preferred embodiments, the basis weight of the synthetic nonwoven fabric material is between about 25 g/sm and about 50 g/sm. In a more preferred embodiment, the basis weight of the synthetic nonwoven fabric material is about 46 g/sm. An outer surface of the synthetic nonwoven material may be provided with a soft calendar pattern for improved adherence, also referred to herein as “adsorption” of the HOCL solution.

In yet another embodiment, the substrate 10 may be a nonwoven fabric material comprising one or more layers of synthetic fibers and/or organic fibers. As previously mentioned, nonwoven materials made of organic fibers are naturally hydrophilic as opposed to nonwoven materials made of normally hydrophobic synthetic fibers yet are more susceptible to environmental and chemical degradation. Consequently, a substrate 10 formed of a nonwoven fabric material made from synthetic fibers and/or organic fibers, such as cellulosic fibers, may further comprise an optional coating of an additive, such as a surfactant, and/or may be infused with the additive to isolate the organic fibers of the substrate 10 from the caustic FAC present in the HOCl solution. If the substrate 10 is formed as a laminate comprising a plurality of layers of nonwoven synthetic and/or organic materials, a suitable water-resistant binding agent or adhesive may be utilized to bond together the individual layers of the substrate 10. Alternatively, the individual layers of the substrate 10 may be bonded together chemically or thermally, for example by ultrasonic welding, or alternatively by hydroentanglement.

In any event, the substrate 10 of synthetic and/or organic materials must be constructed in a manner that sufficiently retains (i.e. holds, carries, clings, adheres or the like) the HOCl solution on the substrate 10, while not permitting the FAC present in the HOCl solution to degrade the structural integrity of the substrate 10. By way of example and not limitation, the outer surfaces of the substrate 10 may have a soft calendar pattern or the like believed to provide surface tension for a liquid solution, such as the sanitizing and/or disinfecting HOCl solution described herein. As used herein, the term “adsorbs” is intended to mean the substrate 10 holds, carriers, clings, adheres or the like the liquid HOCl solution on the outer surface(s) of the substrate 10, for example, as a result of surface tension produced by the substrate material. The term “adsorbs” used herein is contrary to the meaning of the term “absorbs” as commonly understood by those of skill in the art. Specifically, “absorbs” refers to the ability or characteristic of an object or material to take in or soak in a liquid, while “adsorbs” is intended to refer to the ability or characteristic of an object or material to capture, gather or collect a liquid on an outer surface of the object or material.

Regardless, the substrate 10 is loaded with a sanitizing and/or disinfecting solution, indicated generally at 18, and more particularly, a stable HOCl solution. As used herein, the term “loaded” is intended to refer to any suitable means or method for applying the sanitizing and/or disinfecting solution onto the substrate 10. By way of example and not limitation, the sanitizing and/or disinfecting solution 18 may be applied onto the substrate 10 by spraying, coating, dipping, soaking, moistening, pre-moistening or the like. The sanitizing and/or disinfecting solution 18 may be applied during manufacture of the substrate 10, or alternatively, may be applied by a user following manufacture of the substrate 10 (i.e., onto a dry substrate) and prior to or at the time of use. Thus, the term “loaded” is further intended to mean that either the substrate 10 is pre-moistened with the HOCl solution 18 during manufacture, or alternatively, the substrate 10 is moistened with the HOCl solution 18 by the end user prior to or at the time of use. In either case, the substrate 10 is typically moistened up to the point of saturation of the nonwoven fabric material such that the substrate 10 retains the HOCL solution 18 on the exposed outer surfaces of the substrate 10 and thereby acts as a carrier for the HOCL solution 18. For this reason, the substrate 10 pre-moistened or moistened with the HOCl solution 18 is referred to herein as an applicator.

Importantly, the substrate 10 holds the HOCl solution 18 on an outer surface of the substrate 18 yet allows the HOCl solution 18 to be cast off (for example by rapid or vigorous shaking the substrate 10) onto a hard surface to be disinfected and/or sanitized with an appropriate dwell time. It has been found that the surface tension of the invented substrate(s) 10 is sufficient to retain the stable HOCl solution 18 on the otherwise normally hydrophobic substrate 10. In certain embodiments, it is believed that an inner layer 16 of a hydrophilic material, a somewhat hydrophilic material, or even a less hydrophobic material contributes to holding the HOCl solution 18 on the outer surface of one or both of the outer layers 12, 14. In essence, the outer layers 12, 14 of the “loaded” substrate 10 adsorb the stable HOCl solution 18 but do not absorb the stable HOCl solution 18. By way of example and not limitation, the HOCl solution 18 may be produced by the Electrochemically Activated Water (EAW) system and method utilizing a generator comprising an electrolysis cell as disclosed in the U.S. patent application Ser. No. 17/344,951. However, the HOCl solution 18 may be made by any suitable process that produces a stable sanitizing and disinfecting solution at a desired potential of hydrogen (pH) and concentration of FAC for an acceptable shelf-life.

As will be readily appreciated by those skilled in the art, HOCl solutions 18 used to sanitize and disinfect are driven by an inversely proportional relationship between FAC concentration and dwell time. In other words, as the FAC increases the required dwell time decreases, and vice versa. Therefore, effective and stable concentrations of FAC for sanitizing and disinfecting can reasonably range between about 25 parts per million (ppm) and about 750 parts per million (ppm). Furthermore, different concentrations of FAC can be formulated to produce effective HOCl solutions 18, and thus, substrates loaded with an effective HOCl solution 18 having an acceptable shelf-life. Consequently, it is desirable to be able to load the substrate 10 with HOCl solutions 18 having a FAC concentration from about 25 ppm to about 750 ppm to provide for various levels of sanitizing and disinfecting. For example, a FAC concentration of from about 250 ppm to about 600 ppm may be preferred for an effective sanitizing and disinfecting HOCl solution 18, while a FAC concentration of from about 400 ppm to about 550 ppm may be more preferred in other applications. Likewise, a potential of hydrogen (pH) of from about 4.5 pH to about 7.5 pH may be suitable for an effective HOCl solution 18, while from about 5.0 pH to about 7.0 pH may be preferred and about 5.5 pH may be more preferred.

In another aspect, a container is provided for containing and storing the substrates 10 according to the invention. More particularly, a combination container and substrates 10 for use with a stable sanitizing and disinfecting HOCl solution 18 according to the invention is provided. FIG. 2 is a perspective view of an embodiment of a chlorine-impermeable first container 20 for receiving and storing substrates 10 and for subsequently dispensing the sanitizing and disinfecting substrates 10 loaded with a stable liquid HOCl solution 18. In one embodiment, the substrates 10 are loaded with the HOCl solution 18 during manufacture to provide a plurality of pre-moistened sanitizing and disinfecting HOCl applicators in the form of conventional alcohol wipes, commonly referred to merely as “wipes.” Consequently, a container 20 of the type shown and described herein may be referred to as a “wipe flat pack.” Regardless, the sanitizing and disinfecting HOCl applicators or HOCl wipes, indicated generally by reference character 22 in FIG. 2, are formed from the substrates 10 and the HOCl solution 18 previously described.

In the embodiment of FIG. 2, a plurality of sheets of the pre-moistened HOCl wipes 22 are disposed within an interior of the container 20 such that individual wipes 22 may be dispensed from the container 20. The container 20 may comprise a pack or similar housing, receptacle or the like, referred to herein as a pouch 24, defining a generally hollow interior 25 for receiving and containing the pre-moistened HOCl wipes 22. Pouch 24 may be made of an opaque plastic material and/or may be provided with an inner lining, such as a foil film, to prevent transmission of ultraviolet light onto the HOCl wipes 22 through the pouch 24 prior to use. The pouch 24 is sealed during manufacture with a removable and preferably resealable cover 26 that provides access to the HOCl wipes 22 within the interior 25 of the container 20. The individual sheets of wipes 22 may be separated by perforations or may be folded and stacked in a known manner to facilitate removal of each wipe 22 individually from the pouch 24. As a result, container 20 provides a reusable package of pre-moistened (i.e. wetted) HOCl wipes 22 in a convenient form to be used for sanitizing and disinfecting hard surfaces in institutional facilities, commercial businesses, public areas and private spaces.

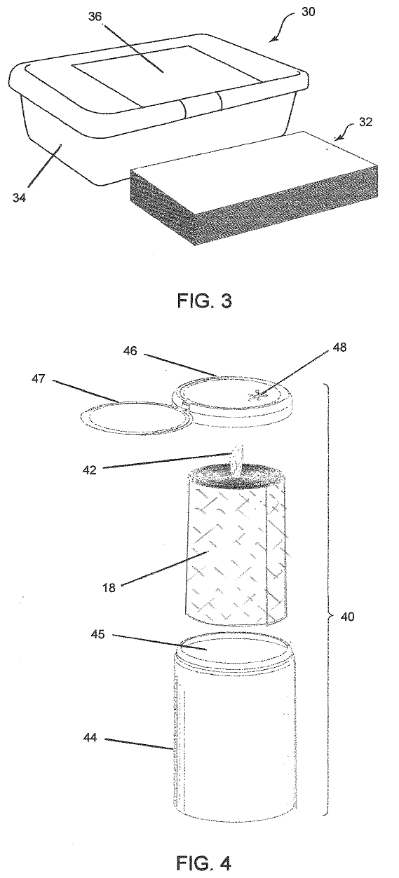

FIG. 3 is a perspective view of an embodiment of a second container 30 for containing and storing substrates 10 according to the invention. More particularly, a combination chlorine-impermeable container for receiving and storing substrates 10 and for subsequently dispensing the substrates 10 for use with a stable sanitizing and disinfecting liquid HOCl solution 18 is provided. The substrates 10 are either loaded with the HOCL solution 18 and stored within an interior of the container, or the substrates 10 are disposed within the interior of the container and then loaded with the HOCl solution 18. In either instance, the loaded substrates 10 are subsequently dispensed from the container for sanitizing and/or disinfecting a hard surface. In one embodiment, the substrates 10 are loaded with the HOCl solution 18 during manufacture to provide a plurality of pre-moistened sanitizing and disinfecting HOCl applicators in the form of conventional alcohol wipes, commonly referred to merely as “wipes.” Consequently, a container 30 of the type shown and described herein may be referred to as a “wipe box.” In another embodiment, the substrates 10 are disposed within the container 30 and the HOCl solution 18 is subsequently introduced into the container 30 such that the substrates 10 are wetted with the HOCl solution 18, for example up to the point of saturation.

Regardless, the sanitizing and disinfecting applicators or wipes, indicated generally by reference character 32 in FIG. 3, are formed from the substrates 10 and the HOCl solution 18 previously described. In the embodiment shown in FIG. 3, a plurality of sheets of the pre-moistened HOCl wipes 32 are contained and stored within the container 30 such that individual wipes 32 may be subsequently dispensed from the container 30. The container 30 may comprise a housing, receptacle or the like, referred to herein as a box 34, defining a generally hollow interior for receiving and containing the pre-moistened HOCl wipes 32. Box 34 may be made of an opaque plastic material and/or may be provided with an inner lining, such as a foil film, to prevent transmission of ultraviolet light onto the HOCl wipes 32 through the box 34. Box 34 is sealed during manufacture with an openable and preferably resealable, for example a hinged, cover 36 that provides access to the HOCl wipes 32 to be dispensed from the container 30. The individual sheets of wipes 32 may be folded and stacked in a known manner to facilitate removal of each wipe 32 individually from the box 34. As a result, container 30 provides a reusable package of pre-moistened (i.e. wetted) HOCl wipes 32 in a convenient form available to be used for sanitizing and disinfecting hard surfaces in institutional facilities, commercial businesses, public areas and private spaces.

FIG. 4 is a perspective view of an embodiment of a third container 40 for the substrates 10 according to the invention. More particularly, a combination chlorine-impermeable container for receiving and storing substrates 10 and for subsequently dispensing the substrates 10 for use with a stable sanitizing and disinfecting liquid HOCl solution 18 is provided. The substrates 10 are either loaded with the HOCl solution 18 and stored within the container to be subsequently dispensed, or the substrates 10 are stored within the container and subsequently loaded with the HOCl solution 18. In one embodiment, the substrates 10 are loaded with the HOCl solution 18 during manufacture to form a perforated roll of pre-moistened sanitizing and disinfecting HOCl applicators. In another embodiment, the substrates 10 in a form similar to a perforated roll of dry cloth or paper towels are disposed within the container 40 and the HOCl solution 18 is subsequently introduced into the container 40 such that the substrates 10 are wetted with the HOCl solution 18, for example up to the point of saturation.

The HOCl solution 18 may be generated and introduced into a container 40 having the perforated roll of the substrates 10 at the time the HOCl solution is generated. Alternatively, the container 40 may be manufactured and provided to the end user and a dry roll of the substrates 10 may be separately manufactured and provided to the end user. In this manner, the end user is able to introduce the dry roll of substrates 10 and the HOCl solution 18 into the container 40 when needed to maximize the shelf-life of the sanitizing and disinfecting HOCl solution 18. The HOCl solution 18 may be pre-packaged in a hermetically sealed receptacle and stored until needed. Alternatively, the HOCl solution 18 may be generated on-site using HOCl generation equipment, for example of the type disclosed in the U.S. patent application Ser. No. 17/344,951 and introduced into the container 40 at the time of use. Regardless, the sanitizing and disinfecting HOCl applicators indicated generally by reference character 42 in FIG. 4 are formed from the substrate 10 and the HOCl solution 18 described herein.

In the embodiment shown in FIG. 4, the substrates 10 (i.e. the HOCl applicators 42) are produced in a continuous sheet form and converted to a perforated roll form. In one embodiment, the perforated roll, also referred to herein as a “log”, comprises a plurality of individual microfiber dry wipes having high surface tension with a length (or width) dimension of at least about six inches and a height dimension of at least about nine inches. Container 40 may comprise a housing, receptacle or the like, referred to herein as a canister 44, defining a generally hollow interior 45 for receiving the perforated roll of dry applicators 42. The canister 44 may be made of an opaque plastic material and/or may be provided with an inner lining, such as a foil film, to prevent transmission of ultraviolet light onto the wipes 42 through the canister 44. Canister 44 is closed during manufacture with a removable and/or openable cover 46 that provides access to the interior 45 of the canister 44 and to the HOCl applicators 42. As illustrated herein, canister 44 and cover 46 may be removably engaged and disengaged by external and internal threads, respectively. In this manner, cover 46 may be removed from canister 44 so that the interior 45 of the canister 44 can subsequently be filled with the HOCl solution 18 to wet the perforated roll of dry substrates 10 with the HOCl solution 18. In one embodiment, the substrates 10 of the HOCl applicators 42 may comprise an outer layer of a wettable polyester fabric material.

As previously described, the substrates 10 may be manufactured and/or the HOCl solution 18 may be generated at an off-site location and provided separately or together with or without container 40. Alternatively, the HOCl solution 18 may be generated off-site and introduced into the container 40 containing the perforated roll of dry substrates 10 either at the time of manufacture or on-site prior to use. Alternatively, the HOCl solution 18 may be generated on-site and introduced into the container 40 containing the perforated roll of dry substrates 18 at the time of use. Still further, the HOCl solution 18 may be provided in a spray cart, spray bottle or liquid dispenser and sprayed or poured onto a dry HOCl applicator 42 dispensed from the container 40. Cover 46 is preferably removable or provided with a hinged portion 47 to open the cover 46 for introducing the HOCl solution 18 into the container 40. Alternatively or in addition, the cover 46 may have a slotted opening 48 for readily dispensing a perforated sheet of wet or dry HOCl applicator 42 without removing the cover 46 from the canister 44. As a result, container 40 provides a reusable package of pre-moistened (i.e. wetted) or dry HOCl applicators 42 in a convenient form available to be used for sanitizing and disinfecting hard surfaces in institutional facilities, commercial businesses, public areas and private spaces.

The substrates 10 loaded with a stable sanitizing and disinfecting HOCl solution 18 may be provided in the form of a single layer or multi-layer wet wipe, wet hand sanitizing wipe, wet mitt, wet cleaning cloth, or wet cleaning pad (each referred to herein as a wet HOCl applicator) for use with or without a cleaning, sanitizing or disinfecting implement, such as a wand, brush, mop, mop head or the like. As used herein, the term “wet” is intended to mean that the substrate 10 is pre-moistened or moistened in situ with the HOCl solution 18, preferably up to the point of the substrate 10 being saturated with the HOCl solution 18. Alternatively, the substrate 10 may be provided in the form of a single layer or multi-layer dry wipe, dry mitt, dry cleaning cloth or dry cleaning pad (each referred to herein as a dry HOCl applicator) for use with or without a cleaning, sanitizing or disinfecting implement, such as a wand, brush, mop, mop head or the like, that is configured to be subsequently moistened with the HOCl solution 18. In the latter instance, the HOCl solution 18 may be generated off-site and delivered with or without the substrate 10, or alternatively, generated on-site at a location where the substrate 10 is to be moistened prior to use so as to maximize the shelf-life of the sanitizing and disinfecting HOCl solution 18. In any of the embodiments or examples provided herein, the substrate 10 may be generically referred to merely as a “wipe” or an “applicator,” subject to any regulatory requirement for approval and/or authorization.

The foregoing detailed description of exemplary embodiments of improved substrates loaded with a stable sanitizing and/or disinfecting solution is merely illustrative of the general concepts and principles of the present invention. Regardless of the foregoing detailed description and illustrated embodiments, various other configurations of the substrates and solutions, as well as reasonable equivalents thereof, will be readily apparent and understood by those of ordinary skill in the art. Accordingly, equivalents to the embodiments described in the written description and shown in the accompanying drawing figures are intended to be encompassed by the broadest reasonable interpretation and construction of the appended claims. Furthermore, as numerous modifications and changes to the exemplary embodiments will readily occur to those skilled in the art, the present invention is not to be limited to the specific configuration, construction, materials, manner of use and operation shown and described herein. Instead, all reasonably predictable and suitable equivalents and obvious modifications to the invention should be determined to fall within the scope of the appended claims given their broadest reasonable interpretation and construction in view of the accompanying written description and drawing figures in view of the combined teachings of the disclosures of the relevant prior art.

That which is claimed is:

1. A substrate comprising:
a multi-ply laminate having a plurality of layers of a nonwoven fabric material, the multi-ply laminate comprising a first outer layer of a normally hydrophobic synthetic polymer and a second outer layer of a normally hydrophobic synthetic polymer;
wherein the first outer layer and/or the second outer layer is configured to receive a stable liquid hypochlorous acid (HOCl) solution such that at least an outer surface of the first outer layer and/or the second outer layer adsorbs the HOCl solution and does not absorb the HOCl solution.

2. The substrate according to claim 1, wherein the HOCl solution has a free available chlorine (FAC) concentration between about 25 ppm and about 750 ppm and a potential of hydrogen (pH) between about 4.5 and about 7.5.

3. The substrate according to claim 1, wherein the normally hydrophobic synthetic polymer of the first outer layer and/or the second outer layer comprises polypropylene (PP) fibers, polyethylene (PE) fibers or polyester (PET) fibers.

4. The substrate according to claim 1, further comprising an inner layer of a synthetic polymer nonwoven fabric material and/or an organic polymer nonwoven fabric material.

5. The substrate according to claim 4, wherein the inner layer is disposed between the first outer layer and the second outer layer.

6. The substrate according to claim 5, wherein the multi-ply laminate is a tri-ply laminate, and wherein the first outer layer comprises Spunbond polypropylene (PP) fibers, the second outer layer comprises Spunbond PP fibers, and the inner layer comprises Meltblown polyester (PET) fibers and/or fluff pulp.

7. The substrate according to claim 1, wherein the first outer layer and the second outer layer are each formed of a Spunbond polypropylene (PP) nonwoven fabric material.

8. The substrate according to claim 7, wherein the outer surface of the first outer layer and/or the second outer layer has a soft calendar pattern.

9. The substrate according to claim 1, wherein the multi-ply laminate comprises an Spunbond-Meltblown-Spunbond (SMS) nonwoven fabric material, and wherein the normally hydrophobic synthetic polymer of the first outer layer and the normally hydrophobic synthetic polymer of the second outer layer comprises polypropylene (PP), polyethylene (PE) and/or polyester (PET) Spunbond fibers, and wherein an inner layer disposed between the first outer layer and the second outer layer comprises Meltblown PET fibers and/or fluff pulp.

10. The substrate according to claim 9, wherein at least the Spunbond fibers of the first outer layer and/or the second outer layer are hydroentangled.

11. The substrate according to claim 1, wherein the normally hydrophobic synthetic polymer of the first outer layer and/or the second outer layer comprises polyester (PET) fibers.

12. The substrate according to claim 11, wherein the synthetic polymer PET fibers of the normally hydrophobic synthetic polymer of the first outer layer and/or the second outer layer are wettable PET fibers with or without a binder system.

13. The substrate according to claim 1, wherein the substrate is configured as an applicator loaded with the stable liquid HOCl solution suitable for applying the HOCl solution to a hard surface to be disinfected and/or sanitized.

14. The substrate according to claim 1, wherein the multi-ply laminate of the nonwoven fabric material is configured as a wipe.

15. The substrate according to claim 1, wherein the multi-ply laminate of the nonwoven fabric material is configured as a perforated roll.

16. A container for the substrate according to claim 1, wherein the container is configured to receive and store the substrate within an interior of the container and to subsequently dispense the substrate from the interior of the container loaded with the HOCl solution or to be loaded with the HOCl solution.

17. A combination container and substrate:
wherein the substrate is a multi-ply laminate comprising a plurality of layers of a nonwoven fabric material, the substrate comprising a first outer layer and a second outer of a normally hydrophobic synthetic polymer, and an inner layer of a synthetic polymer and/or an organic polymer; and
wherein the container has an interior for receiving and storing the substrate, and for subsequently dispensing the substrate from the interior of the container loaded with a stable liquid hypochlorous acid (HOCl) solution or to be loaded with the HOCl solution; and
wherein the HOCl solution adheres to at least an outer surface of the first outer layer and/or an outer surface of the second outer layer without the outer surface of the first outer layer and the outer surface second outer layer absorbing the HOCl solution such that the substrate acts as an applicator that adsorbs the HOCl solution.

18. The combination container and substrate according to claim 17, wherein the normally hydrophobic synthetic polymer of the first outer layer and the second outer layer comprises polypropylene (PP), polyethylene (PE) or polyester (PET).

19. The combination container and substrate according to claim 17, wherein the multi-ply laminate is a Spunbond-Meltblown-Spunbond (SMS) tri-ply laminate, and wherein the normally hydrophobic synthetic polymer of the first outer layer and the second outer comprises Spunbond polypropylene fibers, and wherein the inner layer comprises Meltblown synthetic polymer polyester (PET) fibers and/or Meltblown organic polymer fluff pulp.

20. The combination container and substrate according to claim 17, wherein the container is a chlorine-impermeable package and is configured as a wet pack, a dry pack, a wet box, a dry box, a wet canister or a dry canister.
